(19) United States
(12) Patent Application Publication
(10) Pub. No.: US 2022/0172023 A1
(43) Pub. Date: Jun. 2, 2022

(54) A SYSTEM AND METHOD FOR MEASURING NON-STATIONARY BRAIN SIGNALS

(71) Applicant: AGENCY FOR SCIENCE, TECHNOLOGY AND RESEARCH, Singapore (SG)

(72) Inventors: Haihong ZHANG, Singapore (SG); Huijuan YANG, Singapore (SG); Zhuo ZHANG, Singapore (SG); Chuan Chu WANG, Singapore (SG); Dajiang HE, Singapore (SG); Kai Keng ANG, Singapore (SG)

(21) Appl. No.: 17/599,148

(22) PCT Filed: Mar. 29, 2019
(86) PCT No.: PCT/SG2019/050180

§ 371 Date: Sep. 28, 2021

(51) Int. Cl. G06N 3/04; G06N 3/12

(52) U.S. Cl. CPC G06N 3/04; G06N 3/126

(57) ABSTRACT

Disclosed is a system and method for measuring a non-stationary brain signal. Per the method, the system receives brain signals, extracts one or more features from the brain signals, determines, based on the Receive brain signals extracted one or more features, a super feature set describing dynamic behaviour of the brain signals, and forms a cluster-recurrent-neural-network (CRNN) from one or more samples taken from the super feature set, by formExtract one or more features ing at least one cluster of the one or more samples based on the one or more from the brain signals features, to estimate a brain state of interest in each cluster of brain signals; using a Monte Carlo approach to estimate an a posteriori probability density function of the brain state of interest by applying the CRNN to each cluster of the at least one cluster; and determining the brain state of interest from the estimated density function.

### TECHNICAL FIELD

The present invention relates, in general terms, to a method and system for classifying non-stationary brain signals. In some instances, the method and system determine the concurrent presence of more than one emotional state.

### BACKGROUND

It has become increasingly important to monitor and manage psychological stress (hereafter stress) and emotional signals, to ensure wellbeing.

While some stress can be beneficial, excessive stress is often responsible for negative physiological and psychological consequences such as anxiety, depression, cardiovascular diseases including hypertension.

Emotion is an intense, conscious and psychological experience with a certain degree of pleasure or displeasure. Peoples' response to stimuli vary greatly. However, some peoples' responses differ markedly from the norm. These people often suffer from emotional dysfunction—abnormal emotional response that is poorly modulated, often leading to angry outbursts or behaviour outbursts.

Stress and emotion are highly related to brain activities, and pathological conditions in relation to certain brain dysfunctions. However, it is a rather challenging task to accurately determine the true state of stress and emotion from the measured brain signals. First of all, there is a huge gap between statistical significant patterns discovered by neuroscientists, and any accurate determination of the stress and emotion state determinable continuously, in a real-time fashion, from brain signals.

While neuroscience studies usually depend on a large number of samples/instances to identify specific signals using certain brain imaging or brain signal measurement tools, the identified signals are often limited to statistical value but fail to allow accurate detection in each single instance in the real world applications. Again, this may in part be due to the wide variation in individual response to various stimuli, and variations in conditioning of individuals to stress and particular situations.

Another major cause for the inapplicability of previous neuroscience studies in the identification of emotional states is that brain signals/patterns are highly non-stationary. Both fMRI studies and EEG studies have shown that, for example, even at rest, different functional neural networks in the brain spontaneously fluctuate in activity level. Indeed, the explicit quasistationary phenomena in the activity of large neuronal populations are still largely unknown.

Another issue is that brain states such as emotion and stress often involve a complex network of brain functions and various states. In the current practice of stress and emotion detection, however, data collection and processing methods are very specialized for a particular state. These methods typically do not capture variations in other associated brain states and their dynamics.

It would be desirable to overcome or ameliorate at least one of the abovedescribed problems, or at least to provide a useful alternative.

### SUMMARY

Disclosed herein is a method for measuring a non-stationary brain signal, comprising:
receiving brain signals;
extracting one or more features from the brain signals;
determine, based on the extracted one or more features, a super feature set describing dynamic behaviour of the brain signals;
forming a cluster-recurrent-neural-network (CRNN) from one or more samples taken from the super feature set, by forming at least one cluster of the one or more samples based on the one or more features, to estimate a brain state of interest in each cluster of brain signals;
using a Monte Carlo approach to estimate an a posteriori probability density function of the brain state of interest by applying the CRNN to each cluster of the at least one cluster; and
determining the brain state of interest from the estimated density function.

The brain state of interest may be determined using a maximum likelihood method. The brain state of interest may be any desired format including one taken from a group comprising: categorical, continuous, scalar or multi-variate.

Receiving brain signals may comprise receiving electroencephalogram (EEG) signals. The method may further comprise receiving a further brain signal and determining the further brain signal to be representative of one of the plurality of classes, using the CRNN.

Each feature of the one or more features may be continuous, occurring over a period of time. The super feature set may be determined by calculating one or more of:
averages values in the recent time frames;
linear trends in the recent time frames;
variances in the recent time frames;
frequency components that describes rhythmic fluctuations;
super-fluctuations of the frequency components, i.e. the fluctuations of the rhythmic activities in the raw scores; and
complexity of the raw scores.

Determining a super feature set may comprise forming a super feature set by applying at least one of principal component analysis and independent component analysis to the one or more features. Applying at least one of principal component analysis and independent component analysis may comprise determining a fluctuation of at least one said feature.

Forming a CRNN may comprise taking one or more samples of the super feature set. Forming a CRNN may comprise forming at least one cluster by grouping the one or more samples based on the dynamic behaviour of at least one feature of the one or more features, each cluster hypothetically corresponding to a particular class of the plurality of classes of brain signal, and evaluating a probability that a particular sample of the one or more samples belongs to every cluster. Grouping the one or more samples based on the dynamic behaviour of at least one of the one or more features may comprise grouping the one or more samples based on spectra of the dynamic behaviour of at least one feature of the one or more features. Grouping the one or more samples based on the dynamic behaviour of at least one of the one or more features may instead comprise grouping the one or more samples using k-means clustering based on the dynamic behaviour of at least one feature of the one or more features.

Also disclosed is a method for cross-correlating N concurrent brain states, comprising:
performing the method described above, wherein receiving brain signals comprises receiving brain signals corresponding to trials designed to elicit N-variate responses in the brain signals, each variable of the N-variate responses corresponding to a presence or absence of a respective one of the N concurrent brain states, wherein forming a CRNN comprises clustering the samples into 2^{N} clusters, each cluster being a unique combination of the variables; and
determining a combination of brain states indicated by the further brain signal by applying the CRNN and Monte Carlo approach to estimate an a posteriori probability density function of the combination of brain states using the CRNN and the Monte Carlo approach.

Each brain state may correspond to an emotional state.

In some embodiments, N is 2, and the emotional states are stress/non-stress and happiness/sadness.

Also disclosed herein as a system for measuring a non-stationary brain signal, comprising:
memory; and
at least one processor,
wherein the memory stores instructions that, when executed by the at least one processor, cause the at least one processor to:
   receive brain signals;
   extract one or more features from the brain signals;
   determine, based on the extracted one or more features, a super feature set describing dynamic behaviour of the brain signals;
   form a cluster-recurrent-neural-network (CRNN) from one or more samples taken from the super feature set, by forming at least one cluster of the one or more samples based on the one or more features, to estimate a brain state of interest in each cluster of brain signals;
   use a Monte Carlo approach to estimate an a posteriori probability density function of the brain state of interest by applying the CRNN to each cluster of the at least one cluster; and
   determining the brain state of interest from the estimated density function.

The method may involve determining the brain state of interest using a maximum likelihood method.

The method described above acknowledges the inherent interplays between different brain functions.

Each feature of the one or more features is continuous, occurring over a period of time. The super feature set may be determined by calculating one or more of:
averages values in the recent time frames;
linear trends in the recent time frames;
variances in the recent time frames;
frequency components that describes rhythmic fluctuations;
super-fluctuations of the frequency components, i.e. the fluctuations of the rhythmic activities in the raw scores; and
complexity of the raw scores.

The at least one processor may form the CRNN by taking one or more samples of the super feature set and forming at least one cluster by grouping the one or more samples based on the dynamic behaviour of at least one feature of the one or more features, each cluster hypothetically corresponding to a particular class of the plurality of classes of brain signal, and evaluating a probability that a particular sample of the one or more samples belongs to every cluster.

The at least one processor may group the one or more samples based on the dynamic behaviour of at least one of the one or more features by grouping the one or more samples based on spectra of the dynamic behaviour of at least one feature of the one or more features. The at least one processor may alternatively group the one or more samples based on the dynamic behaviour of at least one of the one or more features by grouping the one or more samples using k-means clustering based on the dynamic behaviour of at least one feature of the one or more features.

The at least one processor may receive brain signals by receiving brain signals corresponding to trials designed to elicit N-variate responses in the brain signals, each variable of the N-variate responses corresponding to a presence or absence of a respective one of the N concurrent brain states, wherein the at least one processor forms a CRNN by clustering the samples into 2^{N} clusters, each cluster being a unique combination of the variables, the at least one processor being configured, by the instructions stored in the memory, to determine a combination of brain states indicated by the further brain signal by classifying the further brain signal as being representative of one of the plurality of classes.

### BRIEF DESCRIPTION OF THE DRAWINGS

Embodiments of the present invention will now be described, by way of non-limiting example, with reference to the drawings in which:

FIG. 1 is a flow chart of a method for measuring a non-stationary brain signal;

FIG. 2 is a system for achieving the method of FIG. 1;

FIG. 3 is a chart showing the correlation between low and high stress and happiness/sadness states; and

FIG. 4 is a chart showing the accuracy of the present invention using a leave one out method of assessment.

### DETAILED DESCRIPTION

The method of the present teachings aims to devise a novel methodology for brain signal detection. The brain signal may be obtained through electromyography (EMG) technology, electroencephalogram technology or any other methodology or technology. The methods described herein may be technology or signal acquisition agnostic.

The present teachings aim to capture various connected brain states as well as the dynamic associations therebetween. Advantageously, the method:
1. Uses a wrapper mechanism. The wrapper mechanism captures and learns the outputs from existing or new brain signal decoding algorithms. These are applied to brain signals acquired through the various technologies mentioned above. The wrapper also learns both short-time and long-time stationarity properties (hereafter also referred to as features) of the output scores, and forms a model of latent modes;
2. Addresses the non-stationarity issue in EEG patterns. To achieve this, the present disclosure proposes a novel Cluster Recurrent Neural Network and a Monte Carlo approach to prediction of brain state. This approach eliminates the need to assume a particular underlying structure in the brain data derived by the technologies mentioned above. Thus various methodologies exist for extracting the brain signal data, each of which can be employed with the present method.

In the present context, a “stationary pattern” refers to the statistics remain the same or consistent across instances (trials), across days, or across subjects. In other words, the statistics remain unchanged and can be distinguished from those that change. The statistics may be a pattern/feature represented as a random variable/vector.

In the statistical learning such as that required to achieve step 108 below, if the statistics properties remain the same, a Bayesian inference/classification principle applied thereto shall stay unchanged. But if the statistics are altered by some underlying shift in the brain mechanism, the Bayesian inference/classification must also adapt. These altering or changing statistics relate to non-stationary patterns.

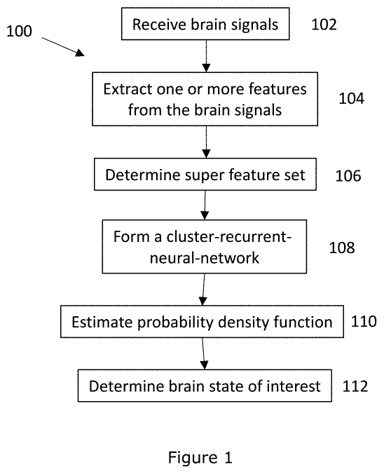

The present methods address the importance of non-stationarity in determining the interplay between different emotional or other brain states. To the end, FIG. 1 provides a method 100 for measuring (e.g. identifying or classifying/categorising) a brain signal, broadly comprising:
102: receiving brain signals;
104: extracting one or more features from the brain signals;
106: determine a super feature set;
108: forming a cluster-recurrent-neural-network (CRNN);
110: estimate a probability density function; and
112: determine a brain state of interest.

In the present context, the brain signals are electroencephalogram (EEG) signals.

The above method 100 addresses the non-stationarity of brain signals, particularly those linked to emotion. The non-stationarity is addressed using cluster structures, and a Bayesian solution is applied to solve the problem of recursively estimating the brain state of interest using the non-stationary data.

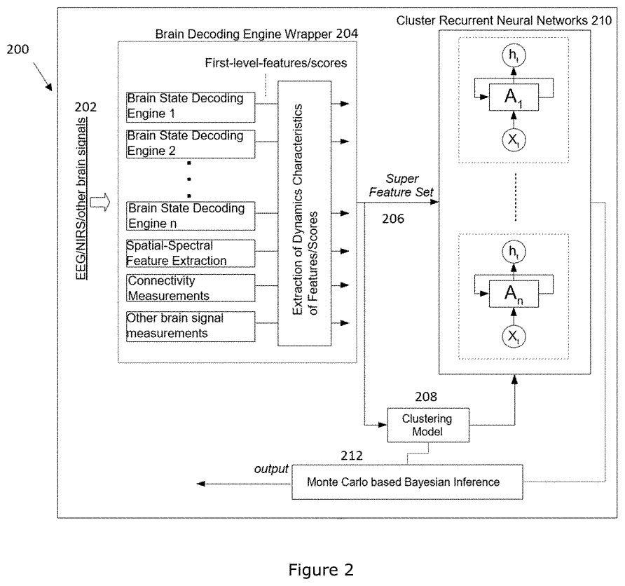

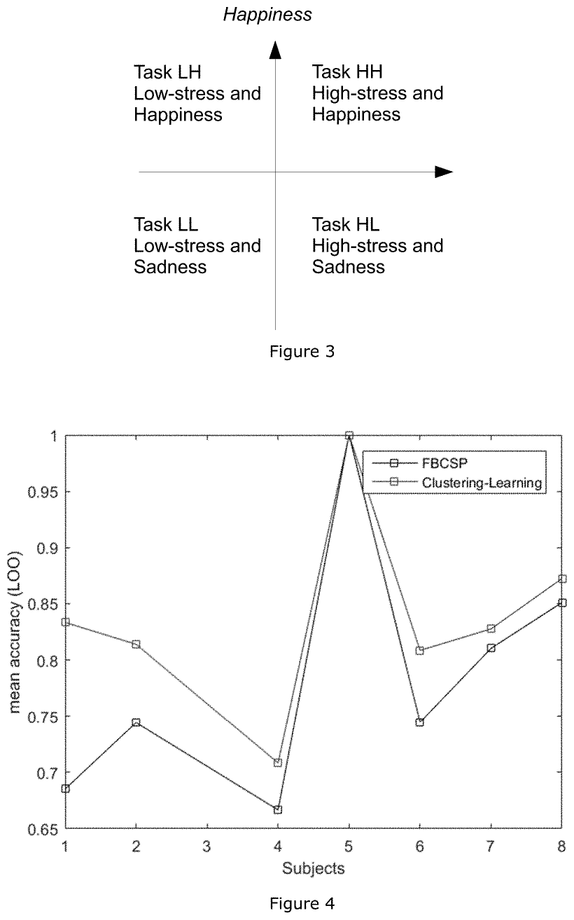

FIG. 2 shows a system or computing system 200 for implementing the method 100. On the left side of FIG. 2 is the input 202 to the system 200. The system 200 receives continuous streams of brain signal measurement data such as EEG/fNIRS (functional near infrared spectroscopy). Other types of inputs may be used, such as electromyography (EMG) inputs and others.

The input 202 may be taken directly from a patient or subject, such as via electrodes attached at various positions on the subject's head to capture EEG signals. Alternatively, the input 202 may be extracted from memory—i.e. in in vivo applications.

The wrapper 204 (entitled “Brain Decoding Engine Wrapper”) extracts one or more features from the brain signals—step 104. The wrapper 204 transforms the incoming data into a rich set of descriptors (features). The features describe the data characteristics relating to both stationary and non-stationary features in the data. Thus, these are first level feature scores such as amplitude, frequency, mean amplitude and frequency and so forth.

To explore various brain functions related to the target signal (or signals) that the system 200 as a whole aims to decode, the system 200 can accommodate various existing or new/future brain state decoding algorithms. In other words, the system 200 and the method 100 employed by it are decoding algorithm agnostic. Each decoding algorithm contains prior knowledge about related brain signals and observations. Those observations may be intuitive or data driven.

The wrapper 204 does not necessarily have to know the exact principles behind each component algorithm, or the training data set that drives it. Such information can nevertheless be incorporated into the system through further extension of the present concepts. In the examples given in the present disclosure, the mechanism is considered to directly plug in and run the algorithms. For example, the method 100 may be in the form of compile code running in a computing device—either hardware or software—largely receiving the output, hereafter referred to as “raw scores”. The raw scores are the one or more features extracted under step 104, that are exhibited in the brain signals received under step 102.

The “raw scores” refer to existing or future individual neurocomputing engines' or modules' outputs.

As mentioned above, the present method 100 and system 200 implementing method 100 examine stationary and non-stationary characteristics of brain signals. To examine both stationary and non-stationary characteristics in each of the continuous raw scores, the system 200 determines, based on the extracted one or more features, a super feature set describing dynamic behaviour of the brain signals—step 106. To do this, the system 200 will compute a variety of features including, but not limited to, one or more from:
Averages values in the recent time frames;
Linear trends in the recent time frames;
Variances in the recent time frames;
Frequency components that describes rhythmic fluctuations;
Super-fluctuations of the frequency components, i.e. the fluctuations of the rhythmic activities in the raw scores; and
Complexity of the raw scores.

Thus, at the output 206 of the wrapper, there could be a large number of features that describe both stationary and non-stationary characteristics in the continuous raw scores. To ease the computational complexity, the system 200 may employ data reduction methods to form the super feature set. These methods include principal component analysis, or independent component analysis.

The principal component analysis converts observations made on the input 202 or raw scores into a set of linearly uncorrelated orthogonal variables, each of which is a principal component. The variables may be ordered in such a way that each principal component has the largest possible variance, given the condition that it is orthogonal to other principal components.

Independent component analysis is a technique to separate out linearly mixed signals to identify artefacts in the signals.

Therefore, the raw scores are transformed into a new set of descriptors of the brain measurements. Whether or not the principal or independent component analysis is used to reduce data complexity, the output of the box 204 is the super feature set. Ideally, each of the one or more features extracted under step 102 will be continuous, enabling its dynamic behaviour to be determined. Similarly, the features of the super feature set, describing the dynamic behaviour of the one or more features, will be continuous—i.e. occurring over a period of time.

This formation of the super feature set is different from feature selection or combination or any conventional transformation. Here the “super feature set” means that a new set of characteristics of each given feature (a variable) is derived. For example, while band powers are widely used, almost ubiquitous EEG features in brain computer interfaces (BCI), the present method 100 may compute dynamics of the band powers to obtain the fluctuation patterns, i.e., fluctuation (specific spectral component) of oscillation levels, or even, fluctuation of fluctuation level of oscillation levels, so on and so forth. It is thus the variation of features over time (e.g. derivative features such as fluctuation mentioned above) that describe the dynamic behaviour of those features, and it is these derivative features that form the super feature set.

The formation of a super feature set is based on the understanding that brain activity and measurement are non-stationary. Instead, they are subject to latent structures. The meaning is two-fold. Firstly, the brain background (aka default network) state can be of multi-modality—that comprises different modes. Each mode may represent an emotion or state such as happiness/sadness, stress/non-stress and so forth.

In each mode, the background brain signal and the mechanism of how the given stimulus or mental task/state may affect the brain signal has different characteristics. Thus each mode shall be better described by a particular computing model. Secondly, variations in the brain signals are time interdependent. This gives rise to a time structure patterns and may also give rise to event sequences appearing in the brain signals—e.g. a particular response is expected on one channel or in one feature, after a different artefact is identified in another channel or feature.

A cluster-recurrent-neural-network (CRNN) is then formed from one or more samples taken from the super feature set—step 108. This is done to estimate a brain state of interest in each cluster of brain signals. The cluster model 208 learns from a pool of data (super feature set samples) and forms a number of clusters, denoted by C₁,C₂, . . . , Cₙ. For a given feature set sample S, it then evaluates the joint probability of the hypothesis that S belongs to every class:

P(Cᵢ|S),I=1, . . . ,n Equation (1)

Thus forming the CRNN involves forming at least one cluster (usually multiple clusters) by grouping the one or more samples based on the dynamic behaviour of at least one feature of the one or more features, each cluster hypothetically corresponding to a particular class of the plurality of classes of brain signal, and evaluating a probability that a particular sample of the one or more samples belongs to every cluster.

The clusters themselves may be determined using spectral clustering algorithms. For example the clusters may be determined by separating individual samples based on whether a particular dynamic feature, or number of dynamic features, falls within a selected spectrum.

The clusters may alternatively be determined using kernel k-means algorithms. In these, samples may be clustered depending on the mean of the cluster that most closely approximates the mean of the respective sample, in one or more of the dynamic features forming the super feature set.

Since this is basically an overall data-driven mechanism, and the number will become a parameter or hyper-parameter that can be learned/tuned.

In an extreme case, there may only be one cluster. The whole mechanism will simply reduce to a conventional neural network. However, the present method 100 was generated to evaluate non-stationarity using the structure of clusters. One single cluster just means that each brain signal of interest is assumed to be stationary, which may work with present teachings but is not the scenario for which method 100 was developed.

The brain state of interest may be represented by a vector of numbers (for example, arousal and valence as two components for emotion). The CRNN then produces a number of pairs of cluster-RNN, and estimates the brain state in each pair—e.g. a tuplet or group of cluster-RNNs of number equal to the number of components of a brain state or concurrent brain states.

The subsequent Monte Carlo approach serves to integrate information from all the cluster-RNN pairs.

For the recurrent neural networks (RNNs) 210, we may consider Long Short-Term Memory (LSTM).

The CRNN may be formed, trained or retained in a variety of ways. In one embodiment, the CRNN is trained by:
Data preparation. In this step, a set of observed and derived brain signal measurement data are built, including feature vector sequence, {Sₜ} sequence, and the associated discrete brain state variable {B}— which can be multi-variate.
Building a clustering model using a pool of feature samples (unsupervised) and the a prior probability of each cluster is calculated. The clustering model shall produce, for a given sample, the normalised likelihood vector I={lᵢ}: Σᵢ lᵢ=1 for that sample.
Learning the probability density function P(S|Cᵢ).
Organising the samples into clusters according to the clustering model.
For each cluster, building a supervised recursive neural network (RNN) that maps every sample (a time-series of feature vectors) in the cluster into the associated class label B.
Running the clustering model and the neural network on the data set. The clustering model and neural network may instead, and preferably, be run on an independent data set. Running the clustering model and neural network collects the data samples where each point consists of the triplet:

{bₖ,{bₖᵢ},Iₖ} Equation (2)
where bₖ is the original (true) brain state variable (vector) of interest of the k-th sample, {bₖᵢ} is the set of predicted brain state variables (vector) predicted by every trained RNN, and Iₖ is the normalized likelihood vector computed by the above clustering model for the given sample.

Running the training code set out above produces a data set {bₖ,{bₖᵢ}, Iₖ}. This data set forms the basis for making online/offline inferences on test/unknown brain signal samples.

Up to this point, the true brain state is unknown though the dynamic characteristics of the brain signals and/or interrelationships between those characteristics is understood. To assess the true brain state for a given time series of super feature vectors or samples therefrom, the two elements in the triplet are computed and obtained—{{bᵢ},}.

From this point, two scenarios of inference are separately considered.

A first scenario (SC1) is akin to block-processing. Each block (a length of time series) of brain signal measurement (EEG/fNIRS, e.g.) is independent of the previous block. One example is a single instance motor imagery task in a given trial. The trial-to-trial interval and the user task therein can be arbitrary, and it would be inappropriate to assume certain dependency between the trials, generally.

The second scenario (SC2) is continuous processing, where each block follows the previous block. One example would be continuous fatigue monitoring. The data blocks consecutively being processed are, for example, with a 1-second shifting time window. There will be much higher likelihood of inter-dependency between the data blocks and the underlying brain states.

Scenario 1—SC1

For SC1, the brain state inference would seek to recover the posterior probability of:

ƒ(b|{bₖ,{bₖᵢ},Iₖ},{{bᵢ},}) Equation (3)

A non-parametric approach may be used to solve this function without assuming its underlying structure of this function. Firstly, a vector q is used to represent the duplet {{bᵢ},1}. Using a Kernel-density estimate, the probability density function can be described by:

$$f ( q \vert \cdot \} = \frac{1}{K} {Σ}_{k} {g}_{h} ( q - {q}_{k} ) \qquad Equation ( 4 )$$

where g is a multi-variate Gaussian function and the covariance matrix is characterized by h.

Since this density function estimate is complex, it is difficult to derive a closed form for the expectation of the brain state variable (vector) b. Note that b is a part of q=[b,{bᵢ},I].

To resolve this issue, Monte Carlo sampling theory is used (at block 212—per step 110) to estimate the expectation of the brain state variable b—this is done by estimating the a posteriori probability density function of the brain state of interest. A number of samples (i.e. “guesses”) of b are then drawn for the given probability density function ƒ(q|·}. The brain state of interest can then be determined—e.g. using a maximum likelihood method—step 112.

The probability density function is assumed to be uniform. Because q is not univariate, it is still an unsolved issue. However, practical approximations are available. For example, Metropolis-Hastings algorithm or the Gibbs Sampling algorithm may be used to obtain samples from ƒ(q|·}. Thus we form a number of qⱼ=[bⱼ,{bᵢ},I], and the expectation of b is simply:

$$\frac{1}{N} {q}_{j} \qquad Equation ( 5 )$$

Scenario 2—SC2

For scenario 2, the time structure between consecutive predictions is considered. This facilitates proper use of the potential interrelationship between samples in a sequence. A Markov Chain Monte Carlo (MCMC) method is proposed for sequentially predicting the brain state variable (vector). Other methods may be applicable.

The skilled person will appreciate the sequential estimation method of the standard MCMC/Particle-Filtering algorithm, and it need not be described in greater detail herein. In addition, reference is made to the above equations in Scenario 1 for the related equations for computing the probability function using the partial samples (duplet {{bᵢ}Iₖ}) and the data points from training the CRNN ({bₖ,{bₖᵢ},Iₖ}).

For sampling from the kernel density estimate ƒ(b|{bₖ,{bₖᵢ},Iₖ}, {{bᵢ},Iₖ}}, the following solution was derived. First, the b by x and the joint vector {{bᵢ},I} by z are denoted. A random-walk Metropolis-Hastings algorithm is then considered and a symmetric Gaussian random-walk proposal function is used as follows:

$$q ( {x}^{( i )} \vert {x}^{( i - 1 )} ) = \frac{1}{{( 2 π )}^{n} \vert {Σ}_{q} \vert} exp [ {( {x}^{( i )} - {x}^{( i - 1 )} )}^{T} {Σ}_{q} ( {x}^{( i )} - {x}^{( i - 1 )} ) ] \qquad Equation ( 6 )$$

Here Σ_{q} is a predefined covariance matrix that determines the randomness of the iterative proposal process. By computing the conditional probability density function for p(x|z), we have the acceptance probability:

$$a ( {x}_{cand} \vert {x}^{( i - 1 )} = min \{ 1 , s ( {x}_{cand} ) \} \\ where \\ s ( {x}_{cand} ) = \frac{{Σ}_{k} \frac{1}{{( 2 π )}^{n} \vert {Σ}_{x \vert z} \vert} exp [ - \frac{1}{2} {( {x}_{cand} - {x}_{k} )}^{T} {Σ}_{x \vert z} ( {x}_{cand} - {x}_{k} ) ]}{{Σ}_{k} \frac{1}{{( 2 π )}^{n} \vert {Σ}_{x \vert z} \vert} exp [ - \frac{1}{2} {( {x}^{( i - 1 )} - {x}_{k} )}^{T} {Σ}_{x \vert z} ( {x}^{( i - 1} ) - {x}_{k} ) ]} \\ {Σ}_{x \vert z} = {Σ}_{zz} - {Σ}_{xz}^{T} {Σ}_{xx}^{- 1} {Σ}_{xz} \\ {x}_{k} = {x}_{k} + {Σ}_{xz} {Σ}_{zz}^{- 1} ( z - zk ) \qquad Equation ( 7 )$$

Following from forming the CRNN per step 108, the resulting model can then be applied to newly received brain signals (which will be understood to constitute the data forming those signals) and the classification of those signals. Thus, the above learning and processing method 100 can be applied to detect, in further brain signals a particular brain state of interest, especially stress and emotion.

Stress, referring to psychological stress, is a feeling of strain and pressure. It is known that positive stress helps improve athletic performance, and also contributes to motivation, adaptation, and reaction to the environment. Excessive amounts of stress, however, may lead to poor performance and adverse psychological and physiological effects. For example, excessive stress links to increased risk of stroke, heart attack, ulcers, and mental disorders such as depression.

In addition, it is known that managing stress properly has a lot to do with managing emotions.

In the body of knowledge on this subject to-date, there appears to be a general lack of a well-articulated way to delineate the complex associations between stress and emotion. Thus, a new data collection protocol has been developed that will enable the study of brain signals in relationship with associations between stress and emotions. The new data protocol seeks to concurrently elicit multiple brain states, so the relationship therebetween can be ascertained.

For a bivariate problem—i.e. two concurrent, different brain states—four different mental tasks are proposed that will lead the brain into different stress-emotion associations. In general, for bivariate brain states, the number of trials attempting to cause each different combination of those states will be 2^{N} where N is the number of states. Thus, receiving brain signals involves receiving signals corresponding to trials designed to elicit N-variate brain signal responses. Each variable of the N-variate responses corresponds to a presence or absence of a respective one of the N concurrent brain states. Thus, the system 200 of FIG. 2 may form a CRNN by clustering the samples into 2^{N} clusters, each cluster being a unique combination of the variables, and the system 200 can then determine a combination of brain states indicated by the further brain signal by classifying the further brain signal as being representative of one of the plurality of classes.

For the current, bivariate problem, the four tasks are named “LH”, “LL”, “HH” and “HL” respectively.

“LH” aims to elicit low-stress and happiness. Autobiographical happy memory recall is recommended for this task.

“LL” aims to elicit low-stress and sadness. Audio-visual presentation of saddening scenes or imagination/recall of saddening events is suggested.

During the task, the subject person needs to keep stress-free by not worrying about consequences.

“HL” aims to elicit high-stress and sadness. There is no readily existing method to achieve this combination, and a new task was thus designed. Public speaking was chosen as the medium to elicit high stress and sadness. Public speaking is usually a high-stress effector. For HL, the task involved delivering a public speech about a saddening experience. To avoid the need to set up an audience, a virtual audience may be created through real or fake tele-conference. Alternatively, a game may be used between an avatar allocated to the subject and a rival's avatar, with the loser being penalized (for example, receiving a cut in an incentive or having to do some difficult tasks). Mental tasks that require significant mental power are not recommended (such as in solving arithmetic challenging questions), as these may create inconsistency in mental workload with LH and LL tasks. To induce sadness, a similar audio-visual presentation of saddening scenes may be played in the background, and, to induce stress, at the same time manipulate the rivalry so that the person will see and expect to lose the game.

“HH” aims to elicit high-stress and happiness. Similar to HL, there is no readily existing method for HH. Public speaking can again be used to elicit stress, but the topic will be about happy experiences. Alternatively, a similar game may be introduced between avatars. However, a happiness-eliciting audio-visual presentation will be played in the background, and, to reduce stress, at the same time the rivalry is manipulated so that the person will see and expect to win the game.

Throughout all the task periods, the subject shall minimize movement and may have eyes closed to avoid eye-blinking artefacts.

User ratings need to be collected to obtain subjective evaluation of the effect of the tasks. Only effectively elicited “HH”, “HL”, “LL” and “LH” task instances will be used for learning the stress-emotion model.

Therefore, constructing the stress-emotion dataset and learning the brain signal detection model will consist of the following steps.
1. Preparation of hardware and software system and the participants contributing stress-emotion.
2. Construct a time series of tasks, which each task is randomly chosen from “HH”, “HL”, “LL” and “LH”. The random selection avoids user bias and leveraging the emotion from one trial into a subsequent trial, and artificially inflating the response to the subsequent trial.
3. Screen the data and subjective ratings and select valid data trials.
4. Assign the bi-variate class labels to the task trials: “HH”: [1 1], “HL”: [1 0], “LL”: [0 0], “LH”: [0 1]. This may involve using subjective ratings for perceived stress and emotion.
5. Perform the training of the clustering-RNN networks. The output of each RNN shall be a bi-variate continuous value.
6. Learn the h function parameters for the Kernel-based density estimate, described in the Scenario 1 above.

For detecting a brain signal to infer the underlying stress-emotion states, a Monte Carlo method may be used per Scenario 1 for trial-based computing (not continuous processing blocks), or the Markov-Chain Monte Carlo method described in Scenario 2 may be used for continuous processing. The particular estimate, for example the expectation, can be derived using the stochastic samples therein.

Thus, FIG. 2 provides a system 200 for classifying a non-stationary brain signal in accordance with method 100. The system 200 may comprise memory —the input 202 may be in or derived from that memory, or may be taken directly from the subject. The system 200 further comprises at least one processor, wherein the memory stores instructions that, when executed by the at least one processor, cause the at least one processor to perform steps 102 to 112. This may involve generating modules and outputs 204 to 212 per FIG. 2.

To numerically validate the proposed technology, the proposed technology was evaluated using existing data, and the results were compared against the state-of-the-art. In the present case, a set of Emotion EEG data were collected in a BCI project involving 8 healthy subjects.

A simplified algorithm was implemented, using a clustering cum a set of classification learning machines, each trained for a specific cluster. The learning machine here uses the Filter-Band-Common-Spatial-Pattern structure. In principal, however, the present methods are not constrained by the particular learning machine. Any learning machine suitable for the particular data type may be used.

To assess the new learning framework's generalization performance, a leave-one-sample-out cross-validation technique was employed and tested on each participant's data individually. The resultant accuracy plot is given in FIG. 4 —the results for Subject 3 are absent because the currently-used clustering technique (k-means) always yields a small cluster size for the 2^{nd} cluster that, unfortunately due to the small sample size ˜50, does not allow full-rank covariance matrix used in the statistical computing algorithm.

For Subject 5, the two algorithms are comparable. For remaining Subjects 1, 2, 4 and 6 to 8, the method 100 produces significantly higher classification accuracy (up to 15% increase). Notably a simplified (non-full implementation) of the proposed technology was used for this initial test. It is envisaged the full implementation will result in potentially higher accuracy.

It will be appreciated that many further modifications and permutations of various aspects of the described embodiments are possible. Accordingly, the described aspects are intended to embrace all such alterations, modifications, and variations that fall within the spirit and scope of the appended claims. Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A method for measuring a non-stationary brain signal, comprising:
receiving brain signals;
extracting one or more features from the brain signals;
determine, based on the extracted one or more features, a super feature set describing dynamic behaviour of the brain signals;
forming a cluster-recurrent-neural-network (CRNN) from one or more samples taken from the super feature set, by forming at least one cluster of the one or more samples based on the one or more features, to estimate a brain state of interest in each cluster of brain signals;
using a Monte Carlo approach to estimate an a posteriori probability density function of the brain state of interest by applying the CRNN to each cluster of the at least one cluster; and
determining the brain state of interest from the estimated density function.

2. The method according to claim 1, wherein receiving brain signals comprises receiving electroencephalogram (EEG) signals.

3. The method according to claim 1, wherein each feature of the one or more features is continuous, occurring over a period of time.

4. The method according to claim 3, wherein the super feature set is determined by calculating one or more of:
averages values in the recent time frames;
linear trends in the recent time frames;
variances in the recent time frames;
frequency components that describes rhythmic fluctuations;
super-fluctuations of the frequency components, i.e. the fluctuations of the rhythmic activities in the raw scores; and
complexity of the raw scores.

5. The method according to claim 1, wherein
determining a super feature set comprises forming a super feature set by applying at least one of principal component analysis and independent component analysis to the one or more features.

6. The method according to claim 5, wherein applying at least one of principal component analysis and independent component analysis comprises determining a fluctuation of at least one said feature.

7. The method according to claim 1, wherein forming a CRNN comprises taking one or more samples of the super feature set.

8. The method according to claim 7, wherein forming a CRNN comprises forming at least one cluster by grouping the one or more samples based on the dynamic behaviour of at least one feature of the one or more features, each cluster hypothetically corresponding to a particular class of the plurality of classes of brain signal, and evaluating a probability that a particular sample of the one or more samples belongs to every cluster.

9. The method according to claim 8, wherein grouping the one or more samples based on the dynamic behaviour of at least one of the one or more features comprises grouping the one or more samples based on spectra of the dynamic behaviour of at least one feature of the one or more features.

10. The method according to claim 8, wherein grouping the one or more samples based on the dynamic behaviour of at least one of the one or more features comprises grouping the one or more samples using k-means clustering based on the dynamic behaviour of at least one feature of the one or more features.

11. The method for cross-correlating N concurrent brain states, comprising:
performing the method of claim 1, wherein receiving brain signals comprises receiving brain signals corresponding to trials designed to elicit N-variate responses in the brain signals, each variable of the N-variate responses corresponding to a presence or absence of a respective one of the N concurrent brain states, wherein forming a CRNN comprises clustering the samples into 2^{N} clusters, each cluster being a unique combination of the variables; and
determining a combination of brain states indicated by the further brain signal by applying the CRNN and Monte Carlo approach to estimate an a posteriori probability density function of the combination of brain states using the CRNN and the Monte Carlo approach.

12. The method according to claim 11, wherein each brain state corresponds to an emotional state.

13. The method according to claim 12, wherein N is 2, and the emotional states are stress/non-stress and happiness/sadness.

14. A system for measuring a non-stationary brain signal, comprising:
memory; and
at least one processor,
wherein the memory stores instructions that, when executed by the at least one processor, cause the at least one processor to:
receive brain signals;
extract one or more features from the brain signals;
determine, based on the extracted one or more features, a super feature set describing dynamic behaviour of the brain signals;
form a cluster-recurrent-neural-network (CRNN) from one or more samples taken from the super feature set, by forming at least one cluster of the one or more samples based on the one or more features, to estimate a brain state of interest in each cluster of brain signals;
use a Monte Carlo approach to estimate an a posteriori probability density function of the brain state of interest by applying the CRNN to each cluster of the at least one cluster; and
determining the brain state of interest from the estimated density function.

15. The system according to claim 14, wherein each feature of the one or more features is continuous, occurring over a period of time.

16. The system according to claim 15, wherein the system determines the system feature set by calculating one or more of:
averages values in the recent time frames;
linear trends in the recent time frames;
variances in the recent time frames;
frequency components that describes rhythmic fluctuations;
super-fluctuations of the frequency components, i.e. the fluctuations of the rhythmic activities in the raw scores; and
complexity of the raw scores.

17. The system according to claim 14, wherein the at least one processor forms the CRNN by taking one or more samples of the super feature set and forming at least one cluster by grouping the one or more samples based on the dynamic behaviour of at least one feature of the one or more features, each cluster hypothetically corresponding to a particular class of the plurality of classes of brain signal, and evaluating a probability that a particular sample of the one or more samples belongs to every cluster.

18. The system according to claim 17, wherein the at least one processor groups the one or more samples based on the dynamic behaviour of at least one of the one or more features by grouping the one or more samples based on spectra of the dynamic behaviour of at least one feature of the one or more features.

19. The system according to claim 17, wherein the at least one processor groups the one or more samples based on the dynamic behaviour of at least one of the one or more features by grouping the one or more samples using k-means clustering based on the dynamic behaviour of at least one feature of the one or more features.

20. The system according to claim 14, wherein the at least one processor receives brain signals by receiving brain signals corresponding to trials designed to elicit N-variate responses in the brain signals, each variable of the N-variate responses corresponding to a presence or absence of a respective one of the N concurrent brain states, wherein the at least one processor forms a CRNN by clustering the samples into 2^{N} clusters, each cluster being a unique combination of the variables, the at least one processor being configured, by the instructions stored in the memory, to determine a combination of brain states indicated by the further brain signal by classifying the further brain signal as being representative of one of the plurality of classes.
